(19) United States
(12) Patent Application Publication
(10) Pub. No.: US 2025/0295770 A1
(43) Pub. Date: Sep. 25, 2025

(54) IgY Antibody Compositions and Methods for Treating Avian Species

(71) Applicant: Mammal Labs LLC, San Jose, CA (US)

(72) Inventors: Vinicius Pereira de Lima Junior, Gainesville, FL (US); Robert A. Burtzlaff, Coos Bay, OR (US); David R. Stevens, Los Gatos, CA (US)

(21) Appl. No.: 19/085,750

(22) Filed: Mar. 20, 2025

Related U.S. Application Data
(60) Provisional application No. 63/568,181, filed on Mar. 21, 2024.

(51) Int. Cl. A61K 39/40; A61K 39/00; A61K 47/36; A61K 47/46; C07K 16/02

(52) U.S. Cl. CPC A61K 39/40; A61K 47/36; A61K 47/46; C07K 16/02; A61K 2039/552

(57) ABSTRACT

A composition and method for therapeutic or prophylactic treatment of birds includes a mixture of IgY antibodies derived from one or more eggs laid by one or more avian species, wherein each of the one or more avian species have been vaccinated with one or more of the plurality of antigens. The mixture of IgY antibodies can be combined with a protective material that includes colostrum.

### RELATED APPLICATION

This application claims the benefit of U.S. Provisional Application Ser. No. 63/568,181 filed Mar. 21, 2024, and entitled IgY Antibody Compositions and Methods for Treating Avian Species, which is hereby incorporated herein by reference.

### FIELD OF INVENTION

The invention provides compositions and methods for treatment of avian species using compositions that include IgY antibodies.

### BACKGROUND OF THE DISCLOSURE

Antibodies, both naturally occurring and their synthetic analogues, are known therapeutic agents. Antibodies bind a portion of the antigen (the antigenic determinant or epitope) with an antibody combining site or antigen binding site. Antibodies are capable of high degrees of specificity enabling targeted application to specific pathogens or toxins.

IgY antibodies from avian eggs have been shown to be effective against pathogens and toxins. Unfortunately, orally delivered antibody therapeutic effectiveness is diminished by passage through the stomach and exposure to gastric acid and digestion enzymes. Improved IgY delivery methods for treatment of avian animals are needed.

### SUMMARY OF THE INVENTION

A composition and method for therapeutic or prophylactic treatment of birds includes a mixture of IgY antibodies derived from one or more eggs laid by one or more avian species, wherein each of the one or more avian species have been vaccinated with one or more of the plurality of antigens. The mixture of IgY antibodies can be combined with a protective material that includes colostrum. In at least some embodiments the protective material further includes colostrum and at least one of an encapsulating material, a polymeric material, a polysaccharide material, and a fatty material.

In another embodiment, an intravenously deliverable composition for therapeutic or prophylactic treatment of animals includes a mixture of IgY antibodies derived from one or more eggs laid by one or more avian species, wherein each of the one or more avian species have been vaccinated with one or more of the plurality of antigens. Also provided is a protective material that slows degradation of the mixture of IgY and other antibodies and is soluble in saline solution. In some embodiments, the mixture of antibodies can include but is not limited to mixtures of IgY with at least one of IgA and IgM.

### BRIEF DESCRIPTION OF THE DRAWINGS

In order that the advantages of the invention will be readily understood, a more particular description of the invention briefly described above will be rendered by reference to specific embodiments illustrated in the appended drawing. Understanding that this drawing depicts only a typical embodiment of the invention and is not therefore to be considered limiting of its scope, the invention will be described and explained with additional specificity and detail through use of the accompanying drawing.

FIG. 1 is a schematic drawing of a technique for producing the disclosed composition; and

FIG. 2 illustrates a representative treatment or prophylactic method.

### DETAILED DESCRIPTION OF THE DISCLOSURE

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “organism” means a form which may have a single cell or multiple cells, and which include bacteria, viruses, parasites, and fungi.

As used herein, “pathogen” means an organism which causes disease or untoward effects in a host and which include bacteria, viruses, and parasites.

As used herein, “toxin” means poison or substance produced by a pathogen and that causes disease or untoward effects in a host.

While this invention is susceptible of embodiment in many different forms, there is shown in the drawings, which will herein be described in detail, specific embodiments with the understanding that the present disclosure is to be considered as an exemplification of the principals of the invention and is not intended to limit the invention to the illustrated embodiments.

We disclose a prophylactic or therapeutic treatment therapeutic composition that includes IgY antibodies derived from eggs laid by chickens or other avian species (e.g. egg-laying hens). These avian species have been immunized against one or more pathogens.

In those embodiments where chickens are used to produce the eggs, the chickens may be any domestic bird of the subspecies Gallus gallus domesticus. Examples include, but are not limited to, the following breeds of Gallus domesticus: Rhode Island Red, Leghorn, Australorp, Lohmann Brown Classic, Sussex, Golden Comet, Marans, Plymouth Rock, Barnevelder, Buff Orpington, Ameraucana, La Brese, and Hamburg. These examples are breeds of chickens which are known to be prolific egg producers. However, other breeds of chickens and other hens of avian species are within the scope of this disclosure. For example, egg laying avians can include ducks, turkeys, geese, or ostriches.

In one embodiment, prior to egg collection, the hens may be immunized with a vaccine comprising at least one antigen which initiates production of antibodies directed against one or more pathogens or toxins. Alternatively or in addition, adjuvants can be added to increase production of naturally present or vaccine induced antibodies. The vaccine may be produced by any method known in the art. Examples include attenuated live vaccines, modified live vaccines, chemically altered vaccines, killed vaccines, toxoid vaccines, DNA vaccines, subunit vaccines, recombinant vaccines, polysaccharide vaccines, and conjugate vaccines. The vaccines may be directed against viruses, bacterial pathogens, parasites, yeasts, or molds. The vaccines may also be directed against toxins, poisons or adhesins produced by pathogens. In some embodiments, the vaccines may include one or more adjuvants which enhance the immunogenicity of the vaccine.

In some embodiments, the avian species may be immunized with vaccines which include a live, wild-type pathogen. Vaccines are typically created using pathogens which have been rendered less virulent (by modifying or killing it) and unlikely to result in clinical illness in the organism receiving the vaccine.

In some embodiments, each member of an avian species may be immunized with a single pathogen. For example, multiple chicken hens may each be immunized with a different single antigen and the eggs from each hen may be combined to result in a product which includes IgY antibodies against different antigens. In other embodiments, a single hen may be immunized with two or more antigens resulting in IgY antibodies against the different antigens present in the same egg.

In some embodiments, pathogens against which the avian species may be immunized include one or more of Salmonella spp., Campylobacter jejuni, Clostridium perfringens, pathogenic E. coli, Avian Rotavirus, Avian Reovirus, and Eimeria spp.

In some embodiments, IgY antibodies can be useful against emergent or undifferentiated pathogen strains with a new combination of features, some of which already reside in other microbes. For example, new combinations of pathogen features result from random mutation, inclusion of DNA from other microbes, or antibiotic-mediated selective evolution. These events create new, highly virulent pathogen strains with limited response to existing medical treatments

In this embodiment, antibodies can be designed to bind to several closely related epitopes that are present within a structurally related cluster of antigens. These antigens may differ markedly in other respects, and may originate from diverse sources, organisms, or species. An inoculant or immunogen can be selected to be a common or preserved component or region of the targeted antigen cluster, while ignoring the variable or distinguishing components or regions of the individual members of the cluster of related antigens. The method involves the preparation a vaccine which will be administered to the avian species and which comprises an appropriate immunogen with characteristics that elicit the production of antibodies that are cross-reactive to desired instances of that epitope, but which are not reactive to other epitopes.

One example of this embodiment includes the production of antitoxin antibodies that are specifically reactive to clusters of structurally related toxins. These example antibodies would have effect without regard to the species originating the toxin. For example, the antibodies raised against the structurally related toxins may be neutralizing antibodies, capable of neutralizing or inactivating the biological activity of the target toxins.

This method can be extended to include any number of toxin clusters, and to include broad-spectrum neutralizing antibodies against mediators of other toxin-like reactions (for example viral toxin-like phenomena). Using these antibodies to prepare a therapeutic as disclosed herein, symptoms and pathology may be managed or prevented without knowledge of the infectious causes, or in cases where there are multiple infectious causes.

Protocols for immunizing the hens of the avian species with the vaccine may be according to those known in the art for initiating antibody production. In an example, the hens may receive two or more vaccinations at least two weeks apart. In some embodiments, the vaccinations may begin when the hens are 18 weeks of age or older. Booster vaccines may be given to the hens 6 months after the first vaccination.

In some embodiments, the vaccines are administered to the hens of the avian species subcutaneously. In other embodiments, the vaccines are administered through intramuscular, oral, intravenous, nasal, spray, or dermal procedures.

After the immunization process, whole shell eggs may be collected from the hens of the avian species. These eggs contain concentrated IgY which bind to the one or more pathogens against which the laying chicken was vaccinated. In other embodiments, the yolk of the eggs may be isolated from the egg whites.

A dehydrated egg powder may be produced from the eggs (either whole shell or isolated yolks) according to procedures known in the art. The drying temperature may be at least 138 degrees F., but not to more than 150 degrees F. which is sufficient to pasteurize the egg and dry to powder within 15 hours. The dehydrated egg product may then be ground to produce a powder suitable for mixing.

In another embodiment, the eggs can be spray dried. In this embodiment, the liquid eggs may be pasteurized at 140 degrees F. immediately prior to spray dry using dedicated food quality process equipment following relevant regulatory guidelines. In some embodiments, high pressure pasteurization can be used. The dried egg product may then be ground produce a powder suitable for mixing.

Additionally, other drying processes, lyophilization, pasteurization, and preservation methods may be used to process the eggs. Furthermore, the antibodies in the eggs may be concentrated, separated, or purified in various ways known in the art. The antibodies produced as disclosed herein may be purified, treated, or retained in the egg material for use in manufacturing the disclosed therapeutic or prophylactic treatment.

In some embodiments, other antibody types, antibody fragments, recombinant antibodies, monoclonal antibodies, polyclonal antibodies, or various antibody mixtures derived from bioreactor or non-avian animal sources can be combined with IgY antibodies. Such antibodies can include IgA, IgM, or other antibodies.

In some embodiments, the colostrum may be bovine colostrum. In some embodiments, the colostrum may be collected from non-hyperimmune ruminants. In some embodiments, non-hyperimmune ruminants may be non-hyperimmune cattle. In some embodiments, the colostrum may comprise of whole colostrum. The colostrum may be dehydrated and ground to a powder using techniques known in the art. Methods described herein for dehydrating the egg preparation may also be used to prepare colostrum powder.

Colostrum serves to slows degradation of the mixture of IgY antibodies in a bird gut and provide additional protective and efficacious attributes to the antibody preparation. Any combination of antibodies may be used within a colostrum matrix, including but not limited to a combination of anti-pathogen, anti-toxin, and anti-adhesin antibodies.

In addition to colostrum, other protein sources may be used as a protective material that slows degradation of the mixture of IgY antibodies in a bird gut and can be mixed with the egg powder preparation. While colostrum includes antibodies derived from the lactating animal, its purpose in this composition is to act as a carrier and to protect the IgY antibodies derived from the egg powder preparation. Examples of other protein sources for use in preparing the protective protein matrix include serum albumin, for example, bovine serum albumin. Dehydrated egg whites may also be used as a protein matrix. While liquid egg whites as found in a chicken egg have approximately 10% protein, a more concentrated protein mixture may be created by dehydrating the egg whites to produce a powder that is added to the egg powder preparation. Protein powder derived from other animal tissues, for example, muscle, gelatin, or collagen of non-hyperimmune animals may also be dehydrated and used to create a powdered protein matrix. Yeast, whey, or whole milk are additional examples of protein sources which may be dehydrated to create a powdered protein matrix.

In yet another embodiment, a protective material other than, or in addition to, colostrum or protein can be used. Such a protective material should slow or reduce degradation of the mixture of IgY antibodies in a bird gut. For example, a protective material can include polymeric material, a polysaccharide material, a fatty material, or their mixtures or combinations. For example, in some embodiments chitosan, alginates, lecithin, cholesterol, or gums can be used. These can be used to coat IgY containing material or be used in various encapsulation methods including but not limited to microcapsules or liposome encapsulation.

In some embodiments, additives or additional substances can combined with a mixture of IgY antibodies for their protective, stabilizing, or other utilities. These can include but are not limited to chemical buffering agents such as sodium bicarbonate, magnesium carbonate, or calcium carbonate. Other pharmaceutical grade buffering agents made by partially neutralizing a weak acid can include trisodium citrate, sodium lactate, trisodium phosphate, or sodium acetate. Other additives can include thermal protection agents such as sucrose, maltose, glycerol, or glycine. In some embodiments, dyes or tracking agents can be added. Other additives, including preservatives or flavorings, may also be included in the final mixture.

In some embodiments one or more of electrolytes, vitamins, and one or more probiotic cultures may also be included in the therapeutic to further support treatment of diarrhea. Probiotics are microbes that are normally found in the gut. They may be bacteria or yeast. When present in proper amounts, probiotic microbes aid in digestion, inhibit growth of pathogenic organisms, and synthesize nutrients. They may also support the host's immune system or have anti-inflammatory activity. In fact, different probiotic strains provide different benefits to the host. It is for at least this reason that probiotic supplements are often provided as a mixture of multiple strains. The mixture may include a plurality of bacteria strains, a plurality of yeast strains, or a plurality of both bacteria and yeast strains. In their absence or in reduced amounts, pathogenic microbes may proliferate in the gut creating an opportunistic infection.

In other embodiments prebiotics can be used to provide nutrients for the probiotic microbes. The protein matrix may act as a prebiotic. Other prebiotics which may be included are fructooligosaccharides (FOS), beet pulp, raw garlic, dandelion greens, wheat dextrin, chicory, fermented vegetables, and other prebiotics known in the art.

In some embodiments, once the preparation of dried egg material including IgY antibodies and the protective material and/or additives are prepared, the two may be mixed to create a powdered substance for using in treating an avian species. The protective protein powder may be provided in proportions of between 1% to 80% by weight of the egg and protein mixture. The powdered egg mixture may be provided in proportions of between 80% and 1% by weight of the egg and protein mixture. In some embodiments, the mixture contains approximately 55% protein or other protective powder and approximately 45% powdered egg preparation by weight. In some embodiments the mixture contains approximately 45% protein or protective powder and approximately 55% powdered egg preparation by weight. In some embodiments, the mixture contains approximately 50% protein or protective powder and approximately 50% powdered egg preparation by weight.

In some embodiments the IgY containing a therapeutic egg powder preparation can administered as an oral, nasal, or mucosal preparation. In other embodiment, the therapeutic egg powder preparation can be prepared for administration as a suppository, intravenous injection, or through any other suitable method. In one embodiment the therapeutic may be administered by sprinkling the dry product onto food which the avian species may then ingest. The therapeutic in dry form may be mixed with water or other ingestible liquid and mixed into or decanted onto food which the avian species may then ingest. The therapeutic in dry form may be mixed with water or other ingestible liquid and administered into the avian species mouth using a syringe or provided for the avian species to drink or administered directly into the avian species stomach through a nasogastric tube. In some embodiments, as will be understood, the combined IgY, protective material, and optional additives can be prepared as a solid, gel, liquid, or aerosol formulation. Such therapeutic or prophylactics can be processed to produce tablets, chewable pills, syrups, elixirs, or aqueous suspensions. Any form known in the art which may be administered to an animal is within the scope of this disclosure. In summary, any method of administering the product into the bird species to be treated is within the scope of this disclosure.

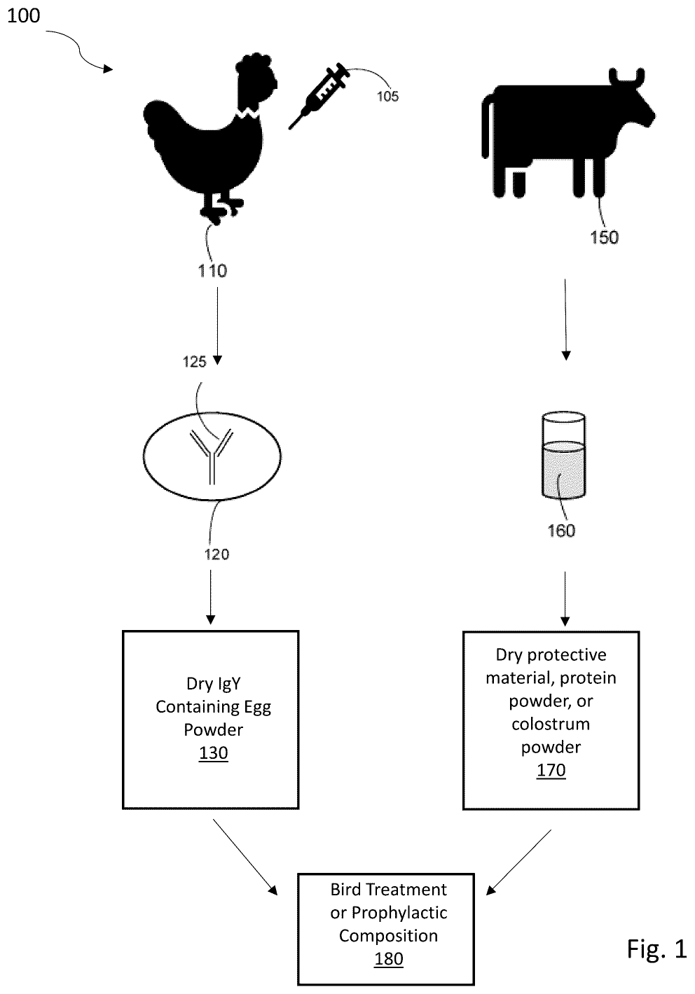

FIG. 1 describes an embodiment 100 of a preparation of the egg powder and protective material. In FIG. 1, chicken 110 is an avian species selected to receive vaccine 105 which includes multiple antigens associated with pathogens or toxins. Chicken 110 then lays egg 120 which contains antibodies, including IgY antibody 125. Many antibodies are present in egg 120 although only antibody 125 is depicted for purposes of clarity. Egg 120 is converted to a dry egg powder using techniques described herein. In this embodiment, the protective material can be a protein matrix prepared from non-hyperimmune bovine colostrum. Lactating cow 150 is a non-hyperimmune animal from which colostrum 160 is collected. Colostrum 160 is converted to a dry colostrum powder 170 using techniques described herein. Dry egg powder 130 and dry colostrum powder 170 are combined in ratios disclosed herein to produce therapeutic or prophylactic 180.

The egg and colostrum (or other matrix protein) mixture may be provided in powdered form. Alternatively, the egg and colostrum mixture may be processed to produce tablets, chewable pills, syrups, elixirs, or aqueous suspensions. Any form known in the art which may be administered orally to an animal is within the scope of this disclosure. Other additives, including preservatives or flavorings, may be included in the final mixture.

The therapeutic may be provided to an avian species in doses that may depend on the animal's body weight, the severity of the disease, and whether the therapeutic is being used to treat existing illness or prophylactically. In an example, a single dose may comprise 3-10 grams of the powdered egg and protein matrix mixture, excluding other additives which may be present in the final product. In some embodiments, a single dose may comprise approximately 5 grams of the powdered egg and protein matrix mixture, excluding other additives which may be present in the final product.

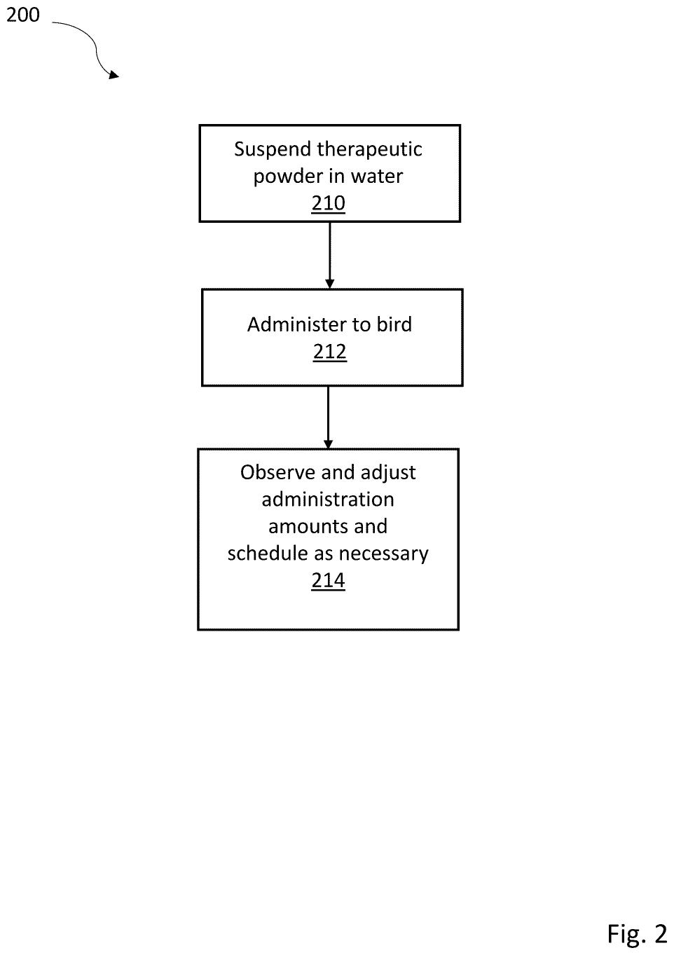

FIG. 2 summarizes an embodiment 200 in which a bird or other avian animal is treated with the disclosed composition. In step 210, a user suspends 5 g of the powdered therapeutic as described herein in 2 ounces of water. The suspended therapeutic is administered orally to a bird suffering from a pathogen or toxin (step 212). The animal is then observed for improvement in symptoms and the administration amounts and schedule are adjusted as necessary (step 214).

It is to be understood that the embodiments of the invention herein described are merely illustrative of the application of the principles of the invention. Reference herein to details of the illustrated embodiments is not intended to limit the scope of the claims, which themselves recite those features regarded as essential to the invention. The following examples are intended to illustrate but not limit the invention.

### Example 1—Avian Vaccine Treatment

Avian hens were vaccinated by oral route and subcutaneous injection with four commercially available animal vaccines. The first was AviPro® Megan® Vac 1 available from Elanco. This vaccine was designed to reduce prevalence of Salmonella typhimurium, Salmonella enteritidis and Salmonella heidelberg in poultry. The vaccine was provided in lyophilized form from attenuated Salmonella typhimurium culture. The vaccine can be administered by spray immunization or in drinking water at. The second vaccine AVERT® NE supplied by Huvepharma and is a live recombinant Salmonella typhimurium strains expressing different clostridial antigens. This vaccine was designed to prevent necrotic enteritis caused by C. perfringens. The third vaccine was Campylobacter fetus-jejuni Bacterin supplied by Colorado Serum (Denver, Colorado). This vaccine is an aqueous suspension of inactivated cultures (killed bacteria) of Campylobacter fetus and Campylobacter jejuni. It contained aluminum hydroxide as an adjuvant and thimerosal as a preservative. The fourth vaccine was Advent Coccidiosis Vaccine made by Huvepharma. This vaccine contains different live attenuated Eimeria species and was designed to prevent incidence of coccidiosis in live poultry.

### Example 2—Bird Treatment Preparation and Application

Following vaccinations, including but limited to those described with respect to Example 1, eggs were collected from hens. A dehydrated egg powder was prepared from the whole shell eggs by drying the eggs and grinding the dehydrated product to produce an egg powder containing IgY. The IgY containing egg powder was combined in a ratio of 50:50 by weight with commercially available bovine colostrum powder which was derived from non-hyperimmune cattle. The mixture was stored at room temperature until reconstituted with water for use.

Ingestion of the described bird treatment preparation will provide prophylactic and active treatment against different Salmonella species, including C. jejuni, C. perfringens and Eimeria spp. pathogens that are commonly found in live poultry. These Salmonella species are part of bird gut microbiome and may cause disease in very intense production environments. Advantageously, treatment can improve the quality and lower contamination of poultry carcasses or eggs. The antibodies present in the mixture of powdered eggs and colostrum will bind and neutralize bacteria, reducing their titers in the intestinal contents, and further reducing the chances of contamination in the final poultry products.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A composition for therapeutic or prophylactic treatment of birds, the composition comprising:
a mixture of IgY antibodies derived from one or more eggs laid by one or more avian species, wherein each of the one or more avian species have been vaccinated with one or more of the plurality of antigens; and
a protective material comprising colostrum.

2. The composition of claim 1, wherein the avian animals comprises at least one of a chicken, duck, turkey, goose, and ostrich.

3. The composition of claim 1, wherein the plurality of antigens are selected from the group consisting of: Salmonella spp., Campylobacter jejuni, Clostridium perfringens, pathogenic E. coli, Avian Rotavirus, Avian Reovirus, and Eimeria spp.

4. The composition of claim 1, wherein the colostrum is derived from a ruminant.

5. The composition of claim 1, wherein the protective material comprises at least 1 percent of the composition by weight.

6. The composition of claim 1, wherein the protective material further comprises an encapsulating material.

7. The composition of claim 1, wherein the protective material further comprises a polymeric material.

8. The composition of claim 1, wherein the protective material further comprises a polysaccharide material.

9. The composition of claim 1, wherein the protective material further comprises a fatty material.

10. A composition for therapeutic or prophylactic treatment of birds, the composition comprising:
a mixture of IgY antibodies derived from one or more eggs laid by one or more avian species, wherein each of the one or more avian species have been vaccinated with one or more of the plurality of antigens; and
a protective material that slows degradation of the mixture of IgY antibodies in a bird gut.

11. The composition of claim 10, wherein the avian animals comprises at least one of a chicken, duck, turkey, goose, and ostrich.

12. The composition of claim 10, wherein the plurality of antigens are selected from the group consisting of: Salmonella spp., Campylobacter jejuni, Clostridium perfringens, pathogenic E. coli, Avian Rotavirus, Avian Reovirus, and Eimeria spp.

13. The composition of claim 10, wherein the protective material comprises colostrum derived from a ruminant.

14. The composition of claim 10, wherein the protective material comprises at least 1 percent of the composition by weight.

15. The composition of claim 10, wherein the protective material further comprises an encapsulating material.

16. The composition of claim 10, wherein the protective material further comprises a polymeric material.

17. The composition of claim 10, wherein the protective material further comprises a polysaccharide material.

18. The composition of claim 10, wherein the protective material further comprises a fatty material.

19. The composition of claim 10, wherein the protective material further comprises colostrum and at least one of an encapsulating material, a polymeric material, a polysaccharide material, and a fatty material.
